(19) United States
(12) Patent Application Publication
(10) Pub. No.: US 2013/0028928 A1
(43) Pub. Date: Jan. 31, 2013

(54) Methods to increase antigenicity of membrane bound polypeptides produced in plants

(71) Applicants: John Howard, Cayucos, CA (US); Celine Hayden, San Luis Obispo, CA (US)

(72) Inventors: John Howard, Cayucos, CA (US); Celine Hayden, San Luis Obispo, CA (US)

(73) Assignee: APPLIED BIOTECHNOLOGY INSTITUTE, San Luis Obispo, CA (US)

(21) Appl. No.: 13/558,874

(22) Filed: Jul. 26, 2012

Related U.S. Application Data
(60) Provisional application No. 61/512,351, filed on Jul. 27, 2011.

(51) Int. Cl. C12P 21/00; A61P 31/14; A61K 39/385

(52) U.S. Cl. 424/193.1; 435/69.3

(57) ABSTRACT

Increased antigenicity of a membrane bound polypeptide produced from plants is provided by reducing fat content of the plant, plant part, or plant tissue producing the polypeptide. Methods and means of producing such plant material are provided. Methods to produce a protective immune response in animals are provided by administering to the animal the plant, plant part of plant tissue which has reduced fat content and which comprises the polypeptide or by administering to the animal an extracted polypeptide produced from such a plant.

### REFERENCE TO RELATED APPLICATION

This application claims priority to previously filed and co-pending application U.S. Ser. No. 61/512,351, filed Jul. 27, 2011, the contents of which are incorporated herein by reference in its entirety.

### STATEMENT AS TO FEDERALLY SPONSORED RESEARCH

Work described herein was funded, at least in part, by the federal government, Grant Number 1 R43 AI068239-01A1 by the National Institute of Health and the United States government as certain rights in the invention.

### SEQUENCE LISTING

The instant application contains a Sequence Listing which has been submitted in ASCII format via EFS-Web and is hereby incorporated by reference in its entirety. Said ASCII copy, created on Jul. 26, 2012, is named AB00015.txt and is 19,187 bytes in size.

### BACKGROUND OF THE INVENTION

Over the past decade, transgenic plants have been successfully used to express a variety of useful proteins. For example, production of proteases in plants has been achieved (See U.S. Pat. No. 6,087,558); along with production of aprotinin in plants (U.S. Pat. No. 5,824,870); and avidin (U.S. Pat. No. 5,767,379). A variety of mammalian bacterial and viral pathogen antigens are included in those proteins that have been successfully produced in plants, such as viral vaccines (U.S. Pat. No. 6,136,320), transmissible gastroenteritis and hepatitis vaccines (U.S. Pat. Nos. 5,914,123 and 6,034,298). These patents, as well as all references cited herein are incorporated herein by reference.

Many of the resulting peptides induced an immunogenic response in mice (Mason et al. (1998) Vaccine 16:13361343; Wigdorovitz et al. (1999) Virology 155:347-353), and humans (Kapusta et al. (1999) FASEB J. 13:1796-1799). After oral delivery, these vaccine candidates were immunogenic and could induce protection. Mice fed a basic diet plus corn expressing recombinant Escherichia coli heat-labile enterotoxin B-subunit (LtB) mounted a dose dependent IgG and IgA response (Streatfield et al. (2001) “Plant based vaccines—unique advances” Vaccine 19:2742-2748.) Some of the first edible vaccine technologies developed include transgenic potatoes expressing hepatitis B, TGEV and Norwalk virus antigens as well as various other viral antigens. (See, e.g., Thanavala et al. (1995) Proc. Natl. Acad. Sci. U.S.A. 92:3358-3361; U.S. Pat. No. 6,136,320; U.S. Pat. No. 6,034,298; U.S. Pat. No. 5,914,123; U.S. Pat. No. 5,612,487 and U.S. Pat. No. 5,484,719; Mason et al., (1996) Proc. Natl. Acad. Sci. 93:5335-5340; “VP1 protein for foot-and-mouth disease” (Wigdorovitz et al (1999) Virology 255:347-353).

The utilization of transgenic plants for vaccine production has several potential benefits over traditional vaccine production methods. First, transgenic plants are usually constructed to express only a small antigenic portion of the pathogen or toxin, eliminating the possibility of infection or innate toxicity of the whole organism and reducing the potential for adverse reactions. Second, since there are no known human or animal pathogens that are able to infect plants, concerns with viral or prion contamination is eliminated. Third, immunogen production in transgenic crops relies on the same established technologies to sow, harvest, store, transport, and process the plant as those commonly used for food crops, making transgenic plants a very economical means of large-scale vaccine production. Fourth, expression of immunogens in the natural protein-storage compartments of plants maximizes stability, minimizes the need for refrigeration and keeps transportation and storage costs low. Fifth, formulation of multicomponent vaccines is possible by blending the seed of multiple transgenic plant lines into a single vaccine. Sixth, direct oral administration is possible when immunogens are expressed in commonly consumed food plants, such as grain, leading to the production of edible vaccines.

To be effective as a vaccine, the protein needs to be produced by the plant in a form that can elicit a protective response to a disease agent. This can be particularly challenging when the protein is a membrane bound protein.

### SUMMARY

Membrane bound polypeptides are expressed in a plant, and the plant, plant part or plant tissue where the polypeptide is capable of producing an antigenic response in an animal when administered to the animal and/or where the polypeptide can be bound by a specific antibody. Such antigenic response is increased by reducing the fat content of the plant, plant part or plant tissue which expresses the polypeptide. With such increased antigenic response an animal may be protected from a disease agent when the polypeptide or plant, plant part or plant tissue is administered to the animal. In an embodiment the plant part may be seed, and in another embodiment may be germ of said seed.

### DESCRIPTION OF DRAWINGS

FIG. 1 shows the sequence of the optimized hepatitis B surface antigen nucleotide sequence used in the experiments along with the Barley Alpha Amylase Signal Sequence (BAASS) in italics and the ATG start codon and stop codon sites in bold (entire sequence is SEQ ID NO: 1, the hepB sequences is SEQ ID NO: 2 and the BAASS sequence is SEQ ID NO: 3).

FIG. 2 shows the sequence of the Belanger et al. 1401 by globulin-1 nucleotide sequence regulatory region (SEQ ID NO: 4) which was used in the experiments below and was followed by 43 extra bases (SEQ ID NO: 11, shown in italics just below the regulatory region sequence). The promoter is bases 1-1386 (SEQ ID NO: 5), the TATA box is at 1354-1360 and the 5′UTR is 1387-1401 (SEQ ID NO: 6).

FIG. 3 is a map of the HBE construct.

FIG. 4 is a map of the HBG construct.

FIG. 5 is the sequence of the extended globulin-1 promoter of U.S. Pat. No. 7,169,967. The entire sequence is SEQ ID NO: 7, with the promoter in regular font (SEQ ID NO: 8), the leader in bold (SEQ ID NO: 9) and the start site codon capitalized.

FIG. 6 is a map of the HBF construct.

FIG. 7 is a graph summarizing the antibody response to mouse feeding trials. Arrows point to days of oral boosting.

### DETAILED DESCRIPTION

A plant-produced polypeptide to be antigenic must be folded properly and produced in a form such that it is capable of being recognized by a specific antibody and/or elicit an animal's protective immune system so that a protective response is generated in the presence of the disease agent. If the animal's body does not recognize the polypeptide as an antigen, no immune reaction will occur and the polypeptide will not be effective in protecting the animal. This is particularly challenging when the polypeptide produced by the plant is a membrane bound protein. Membrane proteins are more difficult to express in transgenic systems than soluble protein. Streatfield et al., (2003) International Journal for Parasitology 33 479-493. Scientists have noted that recombinant expression of integral membrane proteins is considered a major challenge. Eshaghi et al., “An efficient strategy for high-throughput expression screening of recombinant integral membrane proteins” Protein Science, 14:676-683 (2005). Proper processing and folding are among the many steps such proteins require for recognition by a specific antibody and/or producing a protective immune response.

The inventors have discovered it is possible to increase the antigenic nature of the polypeptide, in exposing the site of antigenicity of the membrane bound polypeptide such that it is recognized as an antigen by the animal and an immune or protective response is generated. While it has been widely recognized that adding oil as an adjuvant to a vaccine will increase immune response. See, e.g., Aucouturier et al. (2006) Vaccine Vol. 24, Supp. 2 pp. S44-S45 (International Workshop on Vaccine Adjuvants and Glycoconjugates, Varadero, Cuba Apr. 11-15 2004), the inventors have discovered that it is possible to improve exposure of the antigenic sites by reducing fat content of the plant material producing the polypeptide. Surprisingly, the removal of oil from the plant material producing the polypeptide improved recognition of the antigenic sites. Without wishing to be bound by any theory, the inventors believe that the impact of blocking of receptors by hydrophobic sites of the membrane bound proteins is reduced by reducing oil in the plant material.

The membrane bound proteins in which the invention is useful include any polypeptide that is directed to a membrane-bound organelle and/or the cell membrane, and is not immediately secreted from the cell but remains associated with the membrane for a time. Therefore, membrane-bound proteins are inclusive of external membrane proteins (which are entirely outside of the cell membrane but bound to it by weak molecular attractions, such as ionic, hydrogen, and/or Van der Waals forces) and intrinsic membrane proteins that are embedded in the membrane. Membrane-bound proteins include, for example, integral membrane proteins, transmembrane proteins (which are amphipathic, having hydrophobic and hydrophilic regions and, therefore, having one or more membrane-spanning domains, such as type I and type II transmembrane proteins and multipass transmembrane receptors), peripheral membrane proteins, and lipid-anchored proteins. This includes many viral and glycoproteins. See, e.g., Grisshammer (2006) Current Opinion in Biotechnology 17:337-340 and http://blanco.biomol.uci.edu/mpstruc/listAll/list. When referring to increasing the antigenicity of a polypeptide of a membrane bound protein is meant that the polypeptide has increased recognition as an antigen when compared to the polypeptide produced in plant material in which the fat content has not been reduced. In other words, a polypeptide can be recognized as an antigen by a specific antibody or other agent which binds to or responds to the specific polypeptide/antigen. The antibody or other agent will bind or respond to the antigen to a higher degree with the polypeptide produced from plant material in which fat content is reduced compared to a polypeptide produced in plant material in which fat content is not reduced. B cells and T cells in animals can, for example, recognize a protein as an antigen. In the present context, the polypeptide is antigenic when it produces a protective immune response in an animal or can be bound by an antibody specific for the polypeptide.

One means of testing whether a polypeptide that is antigenic has been produced in such a form is with the Enzyme-linked immunosorbent assay (ELISA). The ELISA has been known since 1971 and detects the presence of an antibody or antigen in a sample. In general, antigens solubilized in a buffer are coated on a surface. When an antibody specific to a polypeptide of the membrane-bound protein is applied over the surface it will bind the antigens. The presence or absence of these antibodies can be demonstrated when the antibody is conjugated to a marker enzyme. Adding the appropriate substrate will detect the amount of bound conjugate which can be quantified. The ELISA test takes many forms. In a typical assay, the antibody is linked with an enzyme and a substance added to convert the enzyme to a detectable signal, usually a color change in the substrate. A common ELISA assay is one which uses biotinylated anti-(protein) polyclonal antibodies and an alkaline phosphatase conjugate. For example, an ELISA used for quantitative determination of levels of a membrane bound polypeptide can be an antibody sandwich assay, which utilizes polyclonal rabbit antibodies obtained commercially. The antibody is conjugated to alkaline phosphatases for detection. In another example, an ELISA assay to detect trypsin or trypsinogen uses biotinylated anti-trypsin or anti-trypsinogen polyclonal antibodies and a streptavidin-alkaline phosphatase conjugate. Obviously there are many variations available to the person skilled in the art, and the detection agent can include fluorogenic, electrochemiluminescent and real time PCR reporters. The specifics of the assay are not critical to the invention, as long as the polypeptide's ability to bind with a specific antibody is assayed. The assay may be qualitative or quantitative.

Another means of testing whether the polypeptide produced is antigenic is to administer the polypeptide to an animal and determine whether an antibody is produced by the animal in response. A further means of testing whether the polypeptide is antigenic is to administer the polypeptide to the animal and determine if a protective immune response is elicited when exposed to the disease-causing agent. The animal may be exposed to the disease causing agent before or after administration of the polypeptide. Measurement and determination of efficacy of any of the compositions and vaccines of the invention may be accomplished by any of the many methods available to one skilled in the art. By way of example, one may measure antibody production in the animal or measure disease morbidity or mortality. A few examples, without intending to be limiting, of diseases caused by membrane-bound polypeptides include hepatitis B, human and simian immunodeficiency virus, rabies virus (RABV), Norwalk virus, Respiratory syncytial virus,

The terms “protecting”, “protection”, “protective immunity” or “protective immune response,” as used herein, are intended to mean that the host animal mounts an active immune response to the vaccine or polypeptides of the present invention, such that upon exposure to the disease challenge, the animal is able to combat the infection. The animal may or may not produce antibodies in response, but the animal will have decreased morbidity or mortality resulting from administration of the vaccine. Thus, a protective immune response will decrease the incidence of morbidity and mortality from exposure to the microorganism among a host animal. The animal will be protected from subsequent exposure to the disease-causing agent. In an embodiment, the animal may be protected by treating the animal which has already been exposed to the disease-causing agent by administration of the vaccine or polypeptide after such exposure. In such an instance there is also shown to be a lessening of morbidity and mortality. When referring to a disease-causing agent it meant the pathogen and is meant to include any such organism causing disease, for example, a virus, bacteria, fungus, or protozoan parasite. Those skilled in the art will understand that in a commercial animal setting, the production of a protective immune response may be assessed by evaluating the effects of vaccination on a group, flock or herd as a whole, e.g., there may still be morbidity and mortality in a minority of vaccinated animals. Furthermore, protection also includes a lessening in severity of any gross or histopathological changes and/or of symptoms of the disease, as compared to those changes or symptoms typically caused by the isolate in similar animals which are unprotected (i.e., relative to an appropriate control). Thus, a protective immune response will decrease the symptoms of the disease, which will vary according to the disease. Disease morbidity and/or mortality are reduced and where there also may be a reduced titer of infection upon exposure to the microorganism.

As used herein, an antibody is defined in terms consistent with that recognized within the art: they are multi-subunit proteins produced by an organism in response to an antigen challenge. Antibodies include monoclonal antibodies and polyclonal antibodies, as well as fragments of such antibodies, including, but not limited to, Fab or F(ab′)hd 2, and Fv fragments.

As used herein, an immunogenically effective amount is employed in a composition for administration to an animal and refers to an amount, which is effective in reducing, eliminating, treating, preventing or controlling the symptoms of the infections, diseases, disorders, or condition.

The invention can also produce a vaccine for administration to the animal. As used herein, the term “vaccine” as used herein refers to a pharmaceutical composition comprising at least one molecule, nucleic acid or polypeptide or fragment thereof that induces a protective response in an animal and possibly, but not necessarily, one or more additional components that enhance the activity of the active component. A vaccine may additionally comprise further components typical to pharmaceutical compositions. A vaccine may comprise one or simultaneously more than one of the elements described above.

The vaccine composition may be introduced into an animal, with a physiologically acceptable vehicle and/or adjuvant. Useful vehicles are well known in the art, and include, e.g., water, buffered water, saline, glycine, hyaluronic acid and the like. The resulting aqueous solutions may be packaged for use as is, or lyophilized, the lyophilized preparation being rehydrated prior to administration, as mentioned above. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like. In an embodiment, the molecule is combined with a binder that assists in associating the molecule with feed, which is particularly useful for oral administration. Such a water resistant binding substance can be any substance having such properties. Examples include, without limitation, agarose or other sugar compounds, albumin, alginate or any similar composition.

In another embodiment, the polypeptide of the invention may be administered with other protective or desirable compounds which may be administered sequentially or progressively or alternately administered simultaneously in an admixture. Single or multiple administrations of the vaccine compositions of the invention can be carried out. Multiple administrations may be required to elicit sufficient levels of immunity.

In referring to administration of the polypeptide of the invention, the polypeptide may be “administered” in any suitable manner, including but not limited to, parenterally, by injection subcutaneously or intramuscularly, into an organ or cavity of the animal, reverse gavage (rectally), and oral, whether per os or ingestion of feed, immersion in a composition or substance containing the polypeptide, as well as transdermal or by gas exchange. The vaccine candidate can be administered by any means which includes, but is not limited to, syringes, nebulizers, misters, needleless injection devices, or microprojectile bombardment gene guns (Biolistic bombardment), via a liposome delivery system, naked delivery system, electroporation, viruses, vectors, viral vectors, or an ingestible delivery system wherein the protective molecules are consumed, for example, in feed or water or in any other suitable manner. Oral or immersion administration is a particular advantage in one embodiment of the invention, in which the plant material may be fed to the animal.

The quantity to be administered depends on the subject to be treated, including, for example, the capacity of the immune system of the individual to mount a protective response. Suitable regimes for initial administration and booster doses are also variable, but may include an initial administration followed by subsequent administrations. The need to provide an effective amount of the protective molecule will also need to be balanced with cost of providing higher amounts of the protective molecule.

The polypeptide of the invention may be administered after extraction from the plant, or plant material comprising the polypeptide of the invention may be administered to the animal. The polypeptide will be extracted after the plant material fat content is reduced, and the plant material will be administered after fat content has been reduced.

If extraction of the polypeptide is desirable, any convenient method may be employed and the invention is not limited by the means of extraction. By way of example without limitation, one method to extract active polypeptides involves homogenizing the entire seed in dry ice and extraction with hexane, extraction with high salt buffer and dialysis against distilled water and precipitating the contaminating globulins. Further purification is accomplished by gel-filtration chromatography, and finally ion-exchange chromatography. See, e.g., PCT WO92/010402 by Willmitzer et al. Protein extraction from biomass can be accomplished by known methods which are discussed, for example, by Heney and Orr, (1981) Anal. Biochem. 114: 92-96.

Reduction of fat content of the plant material can be achieved using any of the many methods available to one skilled in the art and the invention is not limited by the means of fat reduction. As will be evident to one skilled in the art, it does not matter whether the defatting takes place prior to or after introducing the nucleic acid expressing the membrane-bound polypeptide. When referring to fat is meant the lipid or oil content of the plant material. Defatting processes take many forms, including mechanical, using presses or expellers, for example, or chemical compositions. Organic solvents are commonly used to extract oil from plant material by treatment with a solvent which is often a lower carbon alkane, such as propane, butane or hexane. Oil has been traditionally removed from the plant material such as the germ using hexane solvent extraction processes. Yet another means of defatting plant material uses the high pressure process called supercritical fluid extraction. A supercritical fluid is used, any substance at a temperature and pressure above its critical point where distinct liquid and gas phases do not exist. Usually carbon dioxide is used, although any such fluid can be used. In brief, a typical process will move the fluid into a heating zone where it is heated to supercritical conditions and then diffused into a solid matrix and dissolves the material which is to be extracted. See, e.g., R. S. Mohamed and G. A. Mansoori, “Extraction Technology in Food Processing” Food Technology Magazine June, 2002, The World Markets Research Centre, London, UK; Brunner, G. (2005) “Supercritical fluids: technology and application to food processing” Journal of Food Engineering, 67, 21-33. In an embodiment of the invention, it is less desirable to use strong detergents at high levels (an example is Triton X-100 at 1%), in that it may interfere with assays and would reduce the likelihood the plant material could be used as feed in that it would not be suitable for feeding to an animal.

The term plant or plant material or plant part is used broadly herein to include any plant at any stage of development, or to part of a plant, including a plant cutting, a plant cell culture, a plant organ, a plant seed, and a plantlet. Plant seed parts, for example, include the pericarp or kernel, the embryo or germ, and the endoplasm. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. In an embodiment, the tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, the present invention provides plants regenerated from the tissue cultures of the invention.

When using the germ (embryo) of the plant, one can separate the germ from the remainder of the seed and use it as a source of the membrane bound polypeptide. In one embodiment, the promoter driving the polypeptide may be one which is preferentially expressed in the seed, or preferentially expressed in the embryo of the plant, thus even further increasing available polypeptide. Such promoters are discussed below, and methods of using germ as the source of protein are discussed at U.S. Pat. Nos. 7,179,961 and 6,504,085 incorporated herein by reference in their entirety.

The polypeptide of the membrane bound protein will be introduced into a plant. The term introduced in the context of inserting a nucleic acid into a cell, includes transfection or transformation or transduction and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. For a discussion of plant breeding techniques, see Poehlman (1995) Breeding Field Crops. AVI Publication Co., Westport Conn., 4^{th} Edit. Backcrossing methods may be used to introduce a gene into the plants. This technique has been used for decades to introduce traits into a plant. An example of a description of this and other plant breeding methodologies that are well known can be found in references such as Poehlman, supra, and Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent

A “construct” is a package of genetic material inserted into the genome of a cell via various techniques. A “vector” is any means for the transfer of a nucleic acid into a host cell. A vector may be a replicon to which a DNA segment may be attached so as to bring about the replication of the attached segment. A “replicon” is any genetic element (e.g., plasmid, phage, cosmid, chromosome, virus) that functions as an autonomous unit of DNA or RNA replication in vivo, i.e., capable of replication under its own control. In addition to a nucleic acid, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest or produces RNA, and the cassette and restriction sites are designed to ensure insertion of the cassette in the proper reading frame for transcription and translation.

A cell has been “transfected” by exogenous or heterologous DNA or RNA when such DNA or RNA has been introduced inside the cell.

As used herein, the terms nucleic acid or polynucleotide refer to deoxyribonucleotides or ribonucleotides and polymers thereof in either single- or double-stranded form. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally-occurring nucleic acid molecules, which can be isolated from a cell, as well as synthetic molecules, which can be prepared, for example, by methods of chemical synthesis or by enzymatic methods such as by the polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, and mRNA encoded by a gene.

As used herein, a nucleotide segment is referred to as operably linked when it is placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Operably linked elements may be contiguous or non-contiguous. When used to refer to the joining of two protein coding regions, by operably linked it is intended that the coding regions are in the same reading frame. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions.

Nucleic acids of the invention include those that encode an entire polypeptide or fragment thereof. The invention includes not only the exemplified nucleic acids that include the nucleotide sequences as set forth herein, but also nucleic acids that are substantially identical to, correspond to, or substantially complementary to, the exemplified embodiments. For example, the invention includes nucleic acids that include a nucleotide sequence that is at least about 70% identical to one that is set forth herein, more preferably at least 75%, still more preferably at least 80%, more preferably at least 85%, 86%, 87%, 88%, 89% still more preferably at least 90%, 91%, 92%, 93%, 94%, and even more preferably at least about 95%, 96%, 97%, 98%, 99%, 100% identical (or any percentage in between) to an exemplified nucleotide sequence. The nucleotide sequence may be modified as described previously, so long any antigenic polypeptide encoded is capable of inducing the generation of a protective response.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode a protective polypeptide are preferably optimized for expression in a particular host cell (e.g., yeast, mammalian, plant, fungal, and the like) used to produce the polypeptide or RNA.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994). Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, 1984, Proteins).

The isolated variant proteins can be purified from cells that naturally express it, purified from cells that have been altered to express it (recombinant), or synthesized using known protein synthesis methods. For example, a nucleic acid molecule encoding the variant polypeptide is cloned into an expression vector, the expression vector introduced into a host cell and the variant protein expressed in the host cell. The variant protein can then be isolated from the cells by an appropriate purification scheme using standard protein purification techniques.

A protein is comprised of an amino acid sequence when the amino acid sequence is at least part of the final amino acid sequence of the protein. In such a fashion, the protein may be a the original polypeptide, a variant polypeptide and/or have additional amino acid molecules, such as amino acid residues (contiguous encoded sequence) that are naturally associated with it or heterologous amino acid residues/peptide sequences. Such a protein can have a few additional amino acid residues or can comprise several hundred or more additional amino acids.

The variant proteins used in the present invention can be attached to heterologous sequences to form chimeric or fusion proteins. Such chimeric and fusion proteins comprise a variant protein fused in-frame to a heterologous protein having an amino acid sequence not substantially homologous to the variant protein. The heterologous protein can be fused to the N-terminus or C-terminus of the variant protein.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., eds. (1995) Current Protocols in Molecular Biology (Greene Publishing and Wiley-Interscience, New York). Moreover, many expression vectors are commercially available that already encode a fusion moiety (e.g., a GST protein). A variant protein-encoding nucleic acid can be cloned into such an expression vector such that the fusion moiety is linked in-frame to the variant protein.

Polypeptides sometimes contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the variant peptides of the present invention also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The present invention further provides fragments of the variant proteins of the present invention, in addition to proteins and peptides that comprise and consist of such fragments, provided that such fragments act as an antigen and/or provide treatment for and/or protection against infections as provided by the present invention.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1.0 M NaCl, 1% SDS at 37° C., and a wash in 0.1×SSC at 60 to 65° C.

Specificity is also the function of post-hybridization washes, the critical factors being the ionic strength and temperature of the final wash solution. For DNA-DNA hybrids, the Tₘ can be approximated from the equation Tₘ=81.5° C.+16.6 (log M)+0.41(% GC)−0.61(% form.)−500/L, where M is the molarity of monovalent cations, % GC is the percentage of guanosine and cytosine nucleotides in the DNA, % form is the percentage of formamide in the hybridization solution, and L is the length of the hybrid in base pairs (Meinkoth and Wahl, 1984). The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted for sequences of the desired identity to hybridize. For example, if sequences with 90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11 to 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Ausubel et al., eds. (1995) Current Protocols in Molecular Biology (Greene Publishing and Wiley-Interscience, New York) and Sambrook et al., (1989) Molecular Cloning: A Laboratory Manual, 2nd Edition. Cold Spring Harbor Laboratory Press, Plainview, N.Y.

Thus, isolated sequences that have promoter activity and which hybridize under stringent conditions to the promoter sequences disclosed herein, or to fragments thereof, are encompassed by the present invention.

The following terms are used to describe the sequence relationships between two or more nucleic acids or polynucleotides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length promoter sequence, or the complete promoter sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to accurately reflect the similarity to a reference sequence due to inclusion of gaps in the polynucleotide sequence a gap penalty is typically introduced and is subtracted from the number of matches. Methods of alignment of sequences for comparison are well known in the art. Thus, the determination of percent identity between any two sequences can be accomplished using a mathematical algorithm. Optimal alignment of sequences for comparison can use any means to analyze sequence identity (homology) known in the art, e.g., by the progressive alignment method of termed “PILEUP” (Morrison, Mol. Biol. Evol. 14:428-441 (1997), as an example of the use of PILEUP); by the local homology algorithm of Smith & Waterman (Adv. Appl. Math. 2: 482 (1981)); by the homology alignment algorithm of Needleman & Wunsch (J. Mol. Biol. 48:443 (1970)); by the search for similarity method of Pearson (Proc. Natl. Acad. Sci. USA 85: 2444 (1988)); by computerized implementations of these algorithms (e.g., GAP, BEST FIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins, Gene 73: 237-244 (1988); Corpet, Nucleic Acids Res. 16:10881-10890 (1988); Huang, Computer Applications in the Biosciences 8:155-165 (1992); and Pearson, Methods in Mol. Biol. 24:307-331 (1994); Pfam (Sonnhammer, Nucleic Acids Res. 26:322-325 (1998); TreeAlign (Hein, Methods Mol. Biol. 25:349-364 (1994); MEG-ALIGN, and SAM sequence alignment computer programs; or, by manual visual inspection.

Another example of algorithm that is suitable for determining sequence similarity is the BLAST algorithm, which is described in Altschul et al, J. Mol. Biol. 215: 403-410 (1990). The BLAST programs (Basic Local Alignment Search Tool) of Altschul, S. F., et al., (1993) J. Mol. Biol. 215:403-410) searches under default parameters for identity to sequences contained in the BLAST “GENEMBL” database. A sequence can be analyzed for identity to all publicly available DNA sequences contained in the GENEMBL database using the BLASTN algorithm under the default parameters.

Software for performing BLAST analyses is publicly available through the National Center for Biotechnology Information, www.ncbi.nlm.nih.gov/; see also Zhang, Genome Res. 7:649-656 (1997) for the “PowerBLAST” variation. This algorithm involves first identifying high scoring sequence pairs (HSPs) by identifying short words of length W in the query sequence that either match or satisfy some positive valued threshold score T when aligned with a word of the same length in a database sequence. T is referred to as the neighborhood word score threshold (Altschul et al, J. Mol. Biol. 215: 403-410 (1990)). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff, Proc. Natl. Acad. Sci. USA 89:10915-10919 (1992)) alignments (B) of 50, expectation (E) of 10, M=5, N=−4, and a comparison of both strands. The term BLAST refers to the BLAST algorithm which performs a statistical analysis of the similarity between two sequences; see, e.g., Karlin, Proc. Natl. Acad. Sci. USA 90:5873-5787 (1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In an embodiment, GAP (Global Alignment Program) can be used. GAP uses the algorithm of Needleman and Wunsch J. Mol. Biol. 48:443-453 (1970) to find the alignment of two complete sequences that maximizes the number of matches and minimizes the number of gaps. Default gap creation penalty values and gap extension penalty values in the commonly used Version 10 of the Wisconsin Package® (Accelrys, Inc., San Diego, Calif.) for protein sequences are 8 and 2, respectively. For nucleotide sequences the default gap creation penalty is 50 while the default gap extension penalty is 3. Percent Similarity is the percent of the symbols that are similar. Symbols that are across from gaps are ignored. A similarity is scored when the scoring matrix value for a pair of symbols is greater than or equal to 0.50, the similarity threshold. A general purpose scoring system is the BLOSUM62 matrix (Henikoff and Henikoff, Proteins, 17: 49-61 (1993)), which is currently the default choice for BLAST programs. BLOSUM62 uses a combination of three matrices to cover all contingencies. Altschul, J. Mol. Biol. 36: 290-300 (1993), herein incorporated by reference in its entirety and is the scoring matrix used in Version 10 of the Wisconsin Package® (Accelrys, Inc., San Diego, Calif.) (see Henikoff & Henikoff (1989) Proc. Natl. Acad. Sci. USA 89:10915).

As used herein, “sequence identity” or “identity” in the context of two nucleic acid sequences makes reference to the residues in the two sequences that are the same when aligned for maximum correspondence over a specified comparison window.

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

Identity to a sequence used herein would mean a polynucleotide sequence having at least 65% sequence identity, more preferably at least 70% sequence identity, more preferably at least 75% sequence identity, more preferably at least 80% identity, more preferably at least 85% 86%, 87%, 88%, 89%, 90%, 91%, 92%, 93%, 94%, 95%, 96%, 97%, 98%, 99% sequence identity.

The membrane bound polypeptide-encoding nucleic acid molecule may be combined with any number of other components to be introduced into the plant, including combined with another gene of interest to be expressed in the plant. The “gene of interest” refers to a nucleotide sequence that encodes for another desired polypeptide or protein but also may refer to nucleotide sequences that do not constitute an entire gene, and which do not necessarily encode a polypeptide or protein. For example, when used in a homologous recombination process, the nucleic acid molecule may be placed in a construct with a sequence that targets and area of the chromosome in the plant but may not encode a protein. The gene can be used to drive mRNA that can be used for a silencing system, such as antisense, and in that instance, no protein is produced. Means of increasing or inhibiting a protein are well known to one skilled in the art and, by way of example, may include, transgenic expression, antisense suppression, co-suppression methods including but not limited to: RNA interference, gene activation or suppression using transcription factors and/or repressors, mutagenesis including transposon tagging, directed and site-specific mutagenesis, chromosome engineering and, homologous recombination. In the case of use with homologous recombination, no in vivo construct will be required. If desired, the membrane bound polypeptide-encoding nucleic acid molecule or gene of interest can be optimized for plant translation by optimizing the codons used for plants and the sequence around the translational start site for plants. Sequences resulting in potential mRNA instability can also be avoided.

In general, the methods available for construction of recombinant genes, optionally comprising various modifications for improved expression, can differ in detail and any of the methods available to one skilled in the art may be used in the invention. However, conventionally employed methods include PCR amplification, or the designing and synthesis of overlapping, complementary synthetic oligonucleotides, which are annealed and ligated together to yield a gene with convenient restriction sites for cloning, or subcloning from another already cloned source, or cloning from a library. The methods involved are standard methods for a molecular biologist (Sambrook et al., (1989) Molecular Cloning: A Laboratory Manual, 2nd Edition. Cold Spring Harbor Laboratory Press, Plainview, N.Y.).

Once the gene is engineered to contain desired features, such as the desired subcellular localization sequences, it may then be placed into an expression vector by standard methods. The selection of an appropriate expression vector will depend upon the method of introducing the expression vector into host cells. A typical expression vector contains prokaryotic DNA elements coding for a bacterial origin of replication and an antibiotic resistance gene to provide for the growth and selection of the expression vector in the bacterial host; a cloning site for insertion of an exogenous DNA sequence; eukaryotic DNA elements that control initiation of transcription of the exogenous gene (such as the promoter of the invention or another promoter); and DNA elements that control the processing of transcripts, such as transcription termination/polyadenylation sequences. It also can contain such sequences as are needed for the eventual integration of the vector into the plant chromosome.

By “promoter” is meant a regulatory region of DNA capable of regulating the transcription of a sequence linked thereto. It usually comprises a TATA box capable of directing RNA polymerase II to initiate RNA synthesis at the appropriate transcription initiation site for a particular coding sequence. The promoter is the minimal sequence sufficient to direct transcription in a desired manner. The term “regulatory region” is also used to refer to the sequence capable of initiating transcription in a desired manner.

The membrane bound polypeptide-encoding nucleic acid molecule may be used in conjunction with its own or another promoter. In one embodiment, a plant selection marker and the membrane bound polypeptide-encoding nucleic acid molecule or gene of interest can be functionally linked to the same promoter. In another embodiment, they can be functionally linked to different promoters. In yet third and fourth embodiments, the expression vector can contain two or more genes of interest that can be linked to the same promoter or different promoters. For example, one promoter can be used to drive the membrane bound polypeptide-encoding nucleic acid molecule and gene of interest and the selectable marker, or a different promoter used for one or each. These other promoter elements can be those that are constitutive or sufficient to render promoter-dependent gene expression controllable as being cell-type specific, tissue-specific or time or developmental stage specific, or being inducible by external signals or agents. Such elements may be located in the 5′ or 3′ regions of the gene. Although the additional promoter may be the endogenous promoter of a structural gene of interest, the promoter can also be a foreign regulatory sequence. Promoter elements employed to control expression of product proteins and the selection gene can be any plant-compatible promoters. These can be plant gene promoters, such as, for example, the ubiquitin promoter (European patent application no. 0 342 926); the promoter for the small subunit of ribulose-1,5-bis-phosphate carboxylase (ssRUBISCO) (Coruzzi et al., 1984; Broglie et al., 1984); or promoters from the tumor-inducing plasmids from Agrobacterium tumefaciens, such as the nopaline synthase, octopine synthase and mannopine synthase promoters (Velten and Schell, 1985) that have plant activity; or viral promoters such as the cauliflower mosaic virus (CaMV) 19S and 35S promoters (Guilley et al., 1982; Odell et al., 1985), the figwort mosaic virus FLt promoter (Maiti et al., 1997) or the coat protein promoter of TMV (Grdzelishvili et al., 2000). Alternatively, plant promoters such as heat shock promoters for example soybean hsp 17.5-E (Gurley et al., 1986); or ethanol-inducible promoters (Caddick et al., 1998) may be used. See International Patent Application No. WO 91/19806 for a review of illustrative plant promoters suitably employed in the present invention.

A promoter can additionally comprise other recognition sequences generally positioned upstream or 5′ to the TATA box, referred to as upstream promoter elements, which influence the transcription initiation rate. It is recognized that having identified the nucleotide sequences for the promoter region disclosed herein, it is within the state of the art to isolate and identify further regulatory elements in the 5′ region upstream from the particular promoter region identified herein. Thus the promoter region is generally further defined by comprising upstream regulatory elements such as those responsible for tissue and temporal expression of the coding sequence, enhancers and the like.

Tissue-preferred promoters can be utilized to target enhanced transcription and/or expression within a particular plant tissue. When referring to preferential expression, what is meant is expression at a higher level in the particular plant tissue than in other plant tissue. Examples of these types of promoters include seed preferred expression such as that provided by the phaseolin promoter (Bustos et al. (1989) The Plant Cell Vol. 1, 839-853). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, an Ltp1 (See, for example, U.S. Pat. No. 7,550,579), an Ltp2 (Opsahl-Sorteberg, H-G. et al., (2004) Gene 341:49-58 and U.S. Pat. No. 5,525,716), and oleosin genes. See also WO 00/12733, where seed-preferred promoters from end1 and end2 genes are disclosed. Seed-preferred promoters also include those promoters that direct gene expression predominantly to specific tissues within the seed such as, for example, the endosperm-preferred promoter of γ-zein, the cryptic promoter from tobacco (Fobert et al. (1994) “T-DNA tagging of a seed coat-specific cryptic promoter in tobacco” Plant J. 4: 567-577), the P-gene promoter from corn (Chopra et al. (1996) “Alleles of the maize P gene with distinct tissue specificities encode Myb-homologous proteins with C-terminal replacements” Plant Cell 7:1149-1158, Erratum in Plant Cell 1997, 1:109), the globulin-1 promoter from corn (Belanger and Kriz (1991) “Molecular basis for Allelic Polymorphism of the maize Globulin-1 gene” Genetics 129: 863-972 and GenBank accession No. L22344), promoters that direct expression to the seed coat or hull of corn kernels, for example the pericarp-specific glutamine synthetase promoter (Muhitch et al., (2002) “Isolation of a Promoter Sequence From the Glutamine Synthetase₁₋₂ Gene Capable of Conferring Tissue-Specific Gene Expression in Transgenic Maize” Plant Science 163:865-872 and GenBank accession number AF359511) and to the embryo (germ) such as that disclosed at U.S. Pat. No. 7,169,967. When referring to an embryo preferred promoter is meant that it expresses an operably linked sequence to a higher degree in embryo tissue that in other plant tissue. It may express during embryo development, along with expression at other stages, may express strongly during embryo development and to a much lesser degree at other times.

The range of available plant compatible promoters includes inducible promoters. An inducible regulatory element is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the actin of a pathogen or disease agent such as a virus. A plant cell containing an inducible regulatory element may be exposed to an inducer by externally applying the inducer to the cell or plant such as by spraying, watering, heating or similar methods.

Any inducible promoter can be used in the instant invention. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)) Tet repressor from Tn10 (Gatz et al., Mol. Gen. Genet. 227: 229-237 (1991); or from a steroid hormone gene, the transcriptional activity of which is induced by a glucocorticosteroid hormone. Schena et al., Proc. Natl. Acad. Sci. U.S.A. 88: 10421 (1991); the maize GST promoter, which is activated by hydrophobic electrophilic compounds that are used as pre-emergent herbicides; and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Other components of the vector may be included, also depending upon intended use of the gene. Examples include selectable markers, targeting or regulatory sequences, stabilizing or leader sequences, introns etc. General descriptions and examples of plant expression vectors and reporter genes can be found in Gruber, et al., “Vectors for Plant Transformation” in Method in Plant Molecular Biology and Biotechnology, Glick et al eds; CRC Press pp. 89-119 (1993). The selection of an appropriate expression vector will depend upon the host and the method of introducing the expression vector into the host. The expression cassette will also include at the 3′ terminus of the heterologous nucleotide sequence of interest, a transcriptional and translational termination region functional in plants.

In one embodiment, the expression vector also contains a gene encoding a selectable or scoreable marker that is operably or functionally linked to a promoter that controls transcription initiation. Examples of selectable markers include those that confer resistance to antimetabolites such as herbicides or antibiotics, for example, dihydrofolate reductase, which confers resistance to methotrexate (Reiss, (1994) Plant Physiol. (Life Sci. Adv.) 13:143-149; see also Herrera Estrella et al., (1983) Nature 303:209-213; Meijer et al., (1991) Plant Mol. Biol. 16:807-820); neomycin phosphotransferase, which confers resistance to the aminoglycosides neomycin, kanamycin and paromycin (Herrera-Estrella, (1983) EMBO J. 2:987-995, and Fraley et al. (1983) Proc. Natl. Acad. Sci USA 80:4803) and hygro, which confers resistance to hygromycin (Marsh, (1984) Gene 32:481-485; see also Waldron et al., (1985) Plant Mol. Biol. 5:103-108; Zhijian et al., (1995) Plant Science 108:219-227); trpB, which allows cells to utilize indole in place of tryptophan; hisD, which allows cells to utilize histinol in place of histidine (Hartman, (1988) Proc. Natl. Acad. Sci., USA 85:8047); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, (1987), in: Current Communications in Molecular Biology, Cold Spring Harbor Laboratory ed.); and deaminase from Aspergillus terreus, which confers resistance to Blasticidin S (Tamura, (1995) Biosci. Biotechnol. Biochem. 59:2336-2338). Additional selectable markers include, for example, a mutant EPSPV-synthase, which confers glyphosate resistance (Hinchee et al., (1998) BioTechnology 91:915-922), a mutant acetolactate synthase, which confers imidazolinone or sulfonylurea resistance (Lee et al., (1988) EMBO J. 7:1241-1248), a mutant psbA, which confers resistance to atrazine (Smeda et al., (1993) Plant Physiol. 103:911-917), or a mutant protoporphyrinogen oxidase (see U.S. Pat. No. 5,767,373), or other markers conferring resistance to an herbicide such as glufosinate. Examples of suitable selectable marker genes include, but are not limited to, genes encoding resistance to chloramphenicol (Herrera Estrella et al., (1983) EMBO J. 2:987-992); streptomycin (Jones et al., (1987) Mol. Gen. Genet. 210:86-91); spectinomycin (Bretagne-Sagnard et al., (1996) Transgenic Res. 5:131-137,); bleomycin (Hille et al., (1990) Plant Mol. Biol. 7:171-176,); sulfonamide (Guerineau et al., (1990) Plant Mol. Biol. 15:127-136); bromoxynil (Stalker et al., (1988) Science (1986) 242:419-423); glyphosate (Shaw et al., Science 233:478-481); phosphinothricin (DeBlock et al., (1987) EMBO J. 6:2513-2518), and the like. One option for use of a selective gene is a glufosinate-resistance encoding DNA and in one embodiment can be the phosphinothricin acetyl transferase (PAT), maize optimized PAT gene or bar gene under the control of the CaMV 35S or ubiquitin promoters. The genes confer resistance to bialaphos. See, Gordon-Kamm et al., (1990) Plant Cell 2:603; Uchimiya et al., (1993) BioTechnology 11:835; White et al., Nucl. Acids Res. 18:1062, (1990); Spencer et al., 1990) Theor. Appl. Genet. 79:625-631, and Anzai et al., (1989) Mol. Gen. Gen. 219:492. A version of the PAT gene is the maize optimized PAT gene, described at U.S. Pat. No. 6,096,947.

In addition, markers that facilitate identification of a plant cell containing the polynucleotide encoding the marker may be employed. Scorable or screenable markers are useful, where presence of the sequence produces a measurable product and can produce the product without destruction of the plant cell. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al. (1987) The EMBO Journal vol. 6 No. 13 pp. 3901-3907); alkaline phosphatase. Other screenable markers include the anthocyanin/flavonoid genes in general (See discussion at Taylor and Briggs, (1990) The Plant Cell 2:115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al., (1996) Plant Cell 8: 1171-1179; Scheffler et al. (1994) Mol. Gen. Genet. 242:40-48) and maize C2 (Wienand et al., (1986) Mol. Gen. Genet. 203:202-207); the B gene (Chandler et al., (1989) Plant Cell 1:1175-1183), the p1 gene (Grotewold et al, (1991 Proc. Natl. Acad. Sci USA) 88:4587-4591; Grotewold et al., (1994) Cell 76:543-553; Sidorenko et al., (1999) Plant Mol. Biol. 39:11-19); the bronze locus genes (Ralston et al., (1988) Genetics 119:185-197; Nash et al., (1990) Plant Cell 2(11): 1039-1049), among others. Yet further examples of suitable markers include the cyan fluorescent protein (CYP) gene (Bolte et al. (2004) J. Cell Science 117: 943-54 and Kato et al. (2002) Plant Physiol 129: 913-42), the yellow fluorescent protein gene (PhiYFP™ from Evrogen; see Bolte et al. (2004) J. Cell Science 117: 943-54); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); a green fluorescent protein (GFP) gene (Sheen et al., (1995) Plant J. 8(5):777-84); and DsRed where plant cells transformed with the marker gene are red in color, and thus visually selectable (Dietrich et al. (2002) Biotechniques 2(2):286-293). Additional examples include a p-lactamase gene (Sutcliffe, (1978) Proc. Nat'l. Acad. Sci. U.S.A. 75:3737), which encodes an enzyme for which various chromogenic substrates are known (e.g., PADAC, a chromogenic cephalosporin); a xylE gene (Zukowsky et al., (1983) Proc. Nat'l. Acad. Sci. U.S.A. 80:1101), which encodes a catechol dioxygenase that can convert chromogenic catechols; an α-amylase gene (Ikuta et al., (1990) Biotech. 8:241); and a tyrosinase gene (Katz et al., (1983) J. Gen. Microbiol. 129:2703), which encodes an enzyme capable of oxidizing tyrosine to DOPA and dopaquinone, which in turn condenses to form the easily detectable compound melanin. Clearly, many such markers are available to one skilled in the art.

Leader sequences can be included to enhance translation. Various available leader sequences may be substituted or added. Translation leaders are known in the art and include, for example: picornavirus leaders, for example, EMCV leader (encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165 (2):233-8); human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie. (1987) Nucleic Acids Res. 15(8):3257-73); and maize chlorotic mottle virus leader (MCMV) (Lommel et al. (1991) Virology 81:382-385). See also, Della-Cioppa et al. (1987) Plant Physiology 84:965-968.

The expression vector can optionally also contain a signal sequence located between the promoter and the gene of interest and/or after the gene of interest. A signal sequence is a nucleotide sequence, translated to give an amino acid sequence, which is used by a cell to direct the protein or polypeptide of interest to be placed in a particular place within or outside the eukaryotic cell. Many signal sequences are known in the art. See, for example Becker et al., (1992) Plant Mol. Biol. 20:49, Knox, C., et al., “Structure and Organization of Two Divergent Alpha-Amylase Genes from Barley”, Plant Mol. Biol. 9:3-17 (1987), Lerner et al., (1989) Plant Physiol. 91:124-129, Fontes et al., (1991) Plant Cell 3:483-496, Matsuoka et al., (1991) Proc. Natl. Acad. Sci. 88:834, Gould et al., (1989) J. Cell. Biol. 108:1657, Creissen et al., (1991) Plant J. 2:129, Kalderon, et al., (1984) “A short amino acid sequence able to specify nuclear location,” Cell 39:499-509, Steifel, et al., (1990) “Expression of a maize cell wall hydroxyproline-rich glycoprotein gene in early leaf and root vascular differentiation” Plant Cell 2:785-793. When targeting the protein to the cell wall use of a signal sequence is necessary. One example is the barley alpha-amylase signal sequence. Rogers, J. C. (1985) “Two barley alpha-amylase gene families are regulated differently in aleurone cells” J. Biol. Chem. 260: 3731-3738.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson et al. Plant Physiol 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. One skilled in the art will readily appreciate the many options available in expressing a product to a particular organelle. Use of transit peptides is well known (e.g., see U.S. Pat. Nos. 5,717,084; 5,728,925). A protein may be targeted to the endoplasmic reticulum of the plant cell. This may be accomplished by use of a localization sequence, such as KDEL. This sequence (Lys-Asp-Glu-Leu) contains the binding site for a receptor in the endoplasmic reticulum. (Munro et al., (1987) “A C-terminal signal prevents secretion of luminal ER proteins.” Cell. 48:899-907. Retaining the protein in the vacuole is another example. Signal sequences to accomplish this are well known. For example, Raikhel U.S. Pat. No. 5,360,726 shows a vacuole signal sequence as does Warren et al at U.S. Pat. No. 5,889,174. Vacuolar targeting signals may be present either at the amino-terminal portion, (Holwerda et al., (1992) The Plant Cell, 4:307-318, Nakamura et al., (1993) Plant Physiol., 101:1-5), carboxy-terminal portion, or in the internal sequence of the targeted protein. (Tague et al., (1992) The Plant Cell, 4:307-318, Saalbach et al. (1991) The Plant Cell, 3:695-708). Additionally, amino-terminal sequences in conjunction with carboxy-terminal sequences are responsible for vacuolar targeting of gene products (Shinshi et al. (1990) Plant Molec. Biol. 14:357-368).

In addition to a promoter, the expression cassette can include one or more enhancers. By “enhancer” is intended a cis-acting sequence that increases the utilization of a promoter. Such enhancers can be native to a gene or from a heterologous gene. Further, it is recognized that some promoters can contain one or more enhancers or enhancer-like elements. An example of one such enhancer is the 35S enhancer, which can be a single enhancer, or duplicated. See for example, McPherson et al, U.S. Pat. No. 5,322,938. Other methods known to enhance translation can also be utilized, for example, introns, and the like. Other modifications can improve expression, include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The termination region can be native with the promoter nucleotide sequence of the present invention, can be native with the DNA sequence of interest, or can be derived from another source. Convenient termination regions are available from the Ti-plasmid of A. tumefaciens, such as the octopine synthase (MacDonald et al., (1991) Nuc. Acids Res. 19(20)5575-5581) and nopaline synthase termination regions (Depicker et al., (1982) Mol. and Appl. Genet. 1:561-573 and Shaw et al. (1984) Nucleic Acids Research Vol. 12, No. 20 pp 7831-7846 (nos)). Examples of various other terminators include the pin II terminator from the protease inhibitor II gene from potato (An, et al. (1989) Plant Cell 1, 115-122. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Obviously, many variations on the promoters, selectable markers, signal sequences, leader sequences, termination sequences, introns, enhancers and other components of the vector are available to one skilled in the art.

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites, or the like. For this purpose, in vitro mutagenesis, primer repair, restriction digests, annealing, and resubstitutions, such as transitions and transversions, can be involved.

As noted herein, the present invention provides vectors capable of expressing genes of interest. In general, the vectors should be functional in plant cells. At times, it may be preferable to have vectors that are functional in E. coli (e.g., production of protein for raising antibodies, DNA sequence analysis, construction of inserts, obtaining quantities of nucleic acids). Vectors and procedures for cloning and expression in E. coli are discussed in Sambrook et al. (supra).

The transformation vector comprising the sequence of the present invention operably linked to a heterologous nucleotide sequence in an expression cassette, can also contain at least one additional nucleotide sequence for a gene to be cotransformed into the organism. Alternatively, the additional sequence(s) can be provided on another transformation vector.

The method of transformation/transfection is not critical to the instant invention; various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed.

Methods for introducing expression vectors into plant tissue available to one skilled in the art are varied and will depend on the plant selected. Procedures for transforming a wide variety of plant species are well known and described throughout the literature. (See, for example, Miki and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993) Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580.

In one preferred method, the Agrobacterium transformation methods of Ishida et al. (1996) and also described in U.S. Pat. No. 5,591,616, are generally followed, with modifications that the inventors have found improve the number of transformants obtained. The Ishida method uses the A188 variety of maize that produces Type I callus in culture. In one preferred embodiment the Hi II maize line is used which initiates Type II embryogenic callus in culture (Armstrong et al., 1991).

While Ishida recommends selection on phosphinothricin when using the bar or pat gene for selection, another preferred embodiment provides use of bialaphos instead. In general, as set forth in the U.S. Pat. No. 5,591,616, and as outlined in more detail below, dedifferentiation is obtained by culturing an explant of the plant on a dedifferentiation-inducing medium for not less than seven days, and the tissue during or after dedifferentiation is contacted with Agrobacterium having the gene of interest. The cultured tissue can be callus, an adventitious embryo-like tissue or suspension cells, for example. In this preferred embodiment, the suspension of Agrobacterium has a cell population of 10⁶ to 10¹¹ cells/ml and are contacted for three to ten minutes with the tissue, or continuously cultured with Agrobacterium for not less than seven days. The Agrobacterium can contain plasmid pTOK162, with the gene of interest between border sequences of the T region of the plasmid, or the gene of interest may be present in another plasmid-containing Agrobacterium. The virulence region may originate from the virulence region of a Ti plasmid or Ri plasmid. The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, but not an Agrobacterium replication origin, it cannot survive in Agrobacterium without cointegrating with the super binary plasmid. Since the LBA4404 strain is not highly virulent, and has limited application without the super binary plasmid, the inventors have found in yet another embodiment that the EHA101 strain is preferred. It is a disarmed helper strain derived from the hypervirulent A281 strain. The cointegrated super binary/cloning vector from the LBA4404 parent is isolated and electroporated into EHA101, selecting for spectinomycin resistance. The plasmid is isolated to assure that the EHA101 contains the plasmid. EHA101 contains a disarmed pTi that carries resistance to kanamycin. See, Hood et al. (1986).

Further, the Ishida protocol as described provides for growing fresh culture of the Agrobacterium on plates, scraping the bacteria from the plates, and resuspending in the co-culture medium as stated in the U.S. Pat. No. 5,591,616 for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-D, 68.5 g sucrose and 36 g glucose per liter, all at a pH of 5.8. In a further preferred method, the bacteria are grown overnight in a 1 ml culture and then a fresh 10 ml culture is re-inoculated the next day when transformation is to occur. The bacteria grow into log phase, and are harvested at a density of no more than OD₆₀₀=0.5, preferably between 0.2 and 0.5. The bacteria are then centrifuged to remove the media and resuspended in the co-culture medium. Since Hi II is used, medium preferred for Hi II is used. This medium is described in considerable detail by Armstrong and Green (1985). The resuspension medium is the same as that described above. All further Hi II media are as described in Armstrong and Green (1985). The result is redifferentiation of the plant cells and regeneration into a plant. Redifferentiation is sometimes referred to as dedifferentiation, but the former term more accurately describes the process where the cell begins with a form and identity, is placed on a medium in which it loses that identity, and becomes “reprogrammed” to have a new identity. Thus the scutellum cells become embryogenic callus.

In accordance with the present invention, a transgenic plant is produced that contains an introduced nucleic acid molecule encoding the membrane-bound polypeptide.

In a further embodiment, plant breeding can be used to introduce the nucleotide sequences into other plants once transformation has occurred. This can be accomplished by any means known in the art for breeding plants such as, for example, cross pollination of the transgenic plants that are described above with other plants, and selection for plants from subsequent generations which express the amino acid sequence. The plant breeding methods used herein are well known to one skilled in the art. For a discussion of plant breeding techniques, see Poehlman and Sleper (1995). Many crop plants useful in this method are bred through techniques that take advantage of the plant's method of pollination. A plant is self-pollinating if pollen from one flower is transferred to the same or another flower of the same plant. A plant is cross-pollinating if the pollen comes from a flower on a different plant. For example, in Brassica, the plant is normally self-sterile and can only be cross-pollinated unless, through discovery of a mutant or through genetic intervention, self-compatibility is obtained. In self-pollinating species, such as rice, oats, wheat, barley, peas, beans, soybeans, tobacco and cotton, the male and female plants are anatomically juxtaposed. During natural pollination, the male reproductive organs of a given flower pollinate the female reproductive organs of the same flower. Maize plants (Zea mays L.) can be bred by both self-pollination and cross-pollination techniques. Maize has male flowers, located on the tassel, and female flowers, located on the ear, on the same plant. It can self or cross-pollinate.

Pollination can be by any means, including but not limited to hand, wind or insect pollination, or mechanical contact between the male fertile and male sterile plant. For production of hybrid seeds on a commercial scale in most plant species pollination by wind or by insects is preferred. Stricter control of the pollination process can be achieved by using a variety of methods to make one plant pool male sterile, and the other the male fertile pollen donor. This can be accomplished by hand detassling, cytoplasmic male sterility, or control of male sterility through a variety of methods well known to the skilled breeder. Examples of more sophisticated male sterility systems include those described by Brar et al., U.S. Pat. Nos. 4,654,465 and 4,727,219 and Albertsen et al., U.S. Pat. Nos. 5,859,341 and 6,013,859.

Backcrossing methods may be used to introduce the gene into the plants. This technique has been used for decades to introduce traits into a plant. An example of a description of this and other plant breeding methodologies that are well known can be found in references such as Neal (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

The present invention may be used for transformation of any plant species, whether monocotyledonous or dicotyledonous, including but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats (Avena), barley (Hordeum), vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers which may be employed in practicing the present invention include, for example, pines such as loblolly pine (Pinus taeda), slash pine (Pinus elliotii), ponderosa pine (Pinus ponderosa), lodgepole pine (Pinus contotta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

The following is provided by way of illustration of the invention and is not intended to limit the scope of the invention.

### Example 1

### Preparation of Construct

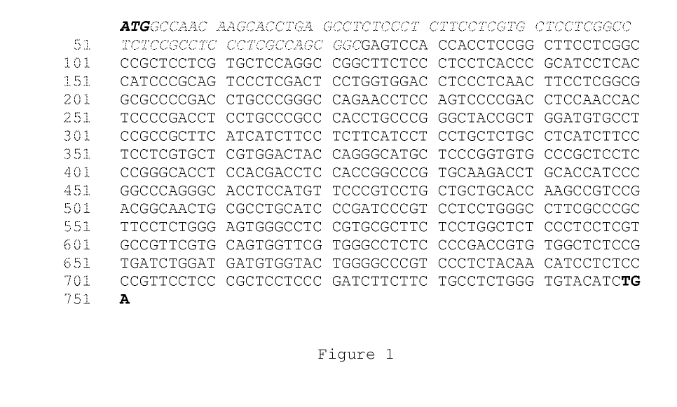

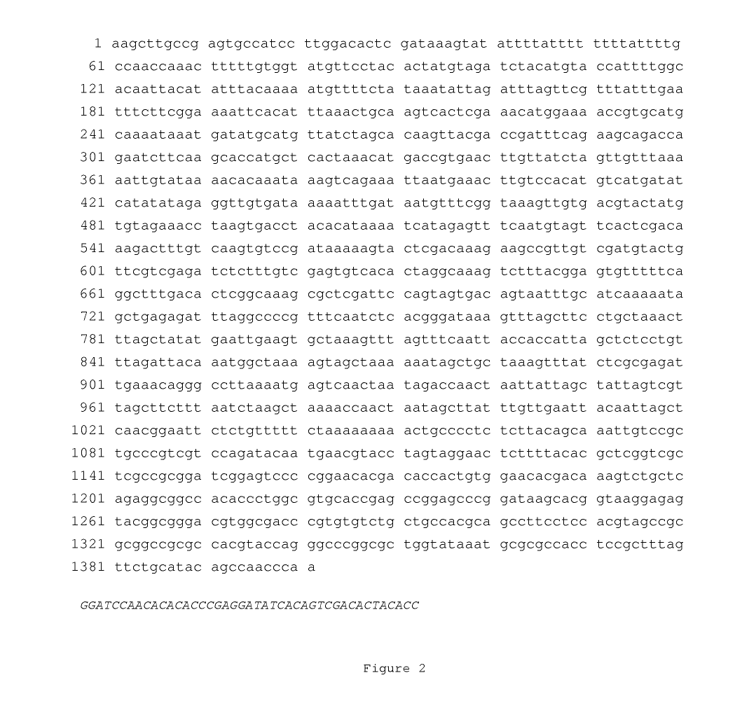

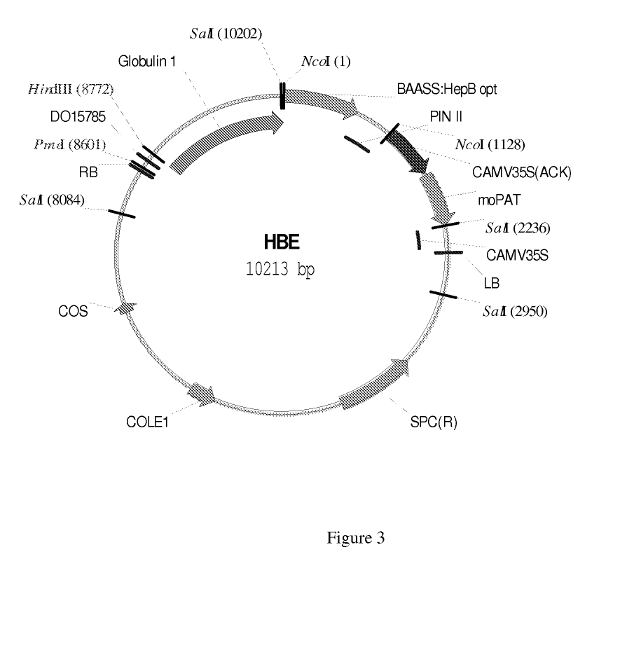

The Hepatitis B surface antigen (HBsAg) sequence, identical to the surface antigen protein sequence available in GenBank accession 562754.1 (adr subtype, small form i.e. S open reading frame without pre-S1 or pre-S2 sequences), was engineered to be codon optimized for expression in maize. See FIG. 1 where the HBsAg optimized sequence is SEQ ID NO: 2 and indicated in regular font with the ATG translation start site in bold. At the N-terminus is a cell-wall targeting sequence, the barley alpha amylase signal sequence, shown in italics and which is SEQ ID NO: 3. (Rogers, J. C. (1985) “Two barley alpha-amylase gene families are regulated differently in aleurone cells” J. Biol. Chem. 260, 3731 3738). The BAASS sequence was fused to HBsAg in order to produce maximally-expressing lines. All HBsAg constructs were built to contain a Pin II (potato proteinase inhibitor II) termination sequence. See An, et al. (1989) “Functional analysis of the 3′ control region of the potato wound-inducible proteinase inhibitor II gene” Plant Cell 1, 115 122. The globulin-1 regulatory region of Belanger et al., supra, of GenBank accession No. L22344 (shown in FIG. 2 in lower case text, SEQ ID NO: 4) was used, followed by an extra 43 bases (shown in FIG. 2 in upper case italics, SEQ ID NO: 11. These extra bases are not relevant to the regulatory region and are believed to be a downstream portion on chromosome 1 of the maize gene, which may originate from a retrotransposon. A selectable marker employed in the construct is the maize optimized PAT sequence providing resistance to the herbicide glufosinate. See, Gordon-Kamm et al., (1990) Plant Cell 2:603; Uchimiya et al., (1993) BioTechnology 11:835; White et al., Nucl. Acids Res. 18:1062, (1990); Spencer et al., (1990) Theor. Appl. Genet. 79:625-631, and Anzai et al., (1989) Mol. Gen. Gen. 219:492. A version of the PAT gene is the maize optimized PAT gene, described in U.S. Pat. No. 6,096,947.) The resulting plasmid HBE map is shown in FIG. 3.

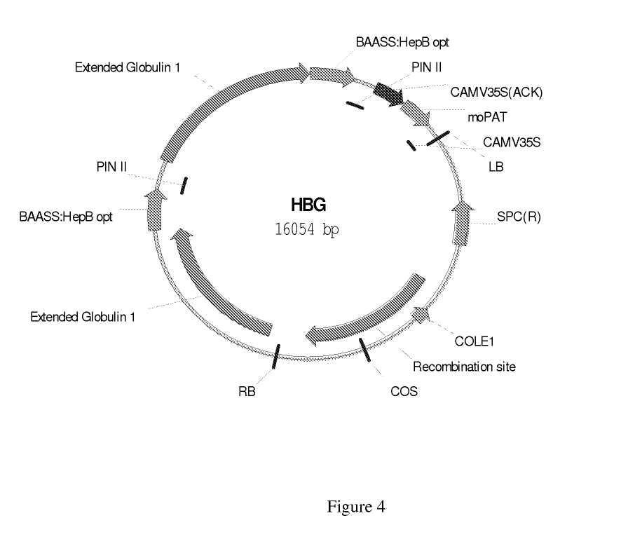

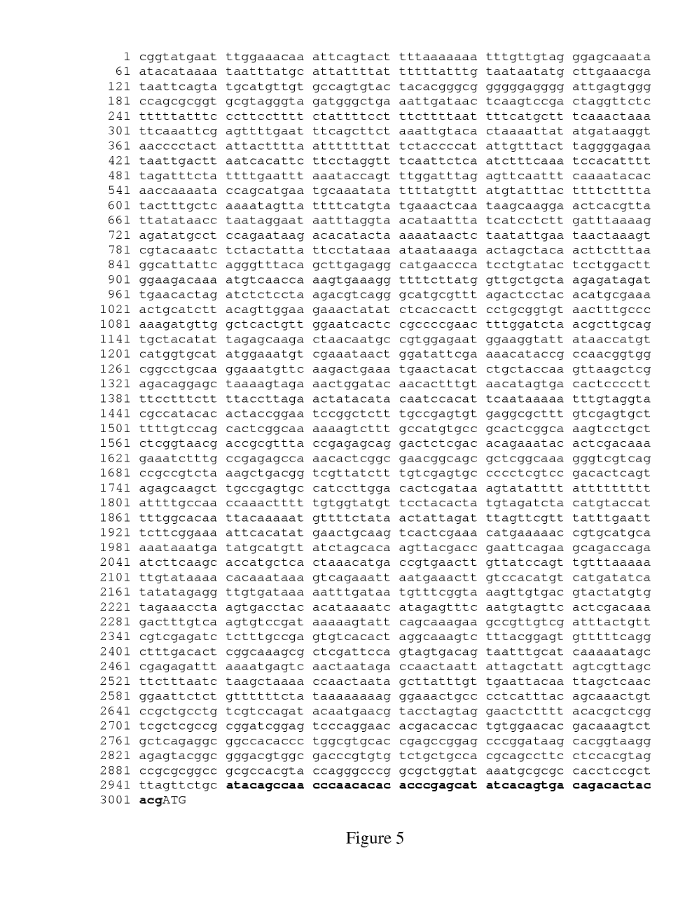

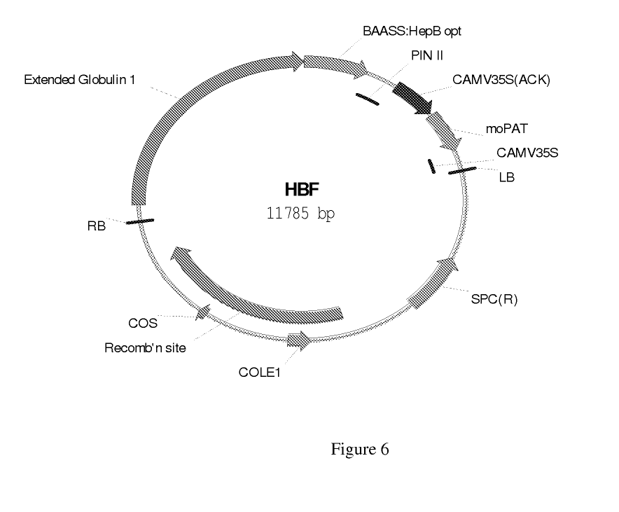

HBG was constructed using the extended globulin-1 promoter of U.S. Pat. No. 7,169,967, incorporated herein by reference and see FIG. 5. The nucleotide sequence used here (SEQ ID NO: 10) is the same as that of the '967 patent, with one base change at 3003 of FIG. 5, the base directly preceding the ATG start codon where a C instead of a G was used (to incorporate the NcoI restriction site at the start codon). See FIG. 4 for the plasmid map. Two plant transcription units (PTUs), each consisting of a promoter, protein coding sequence, and termination sequence, were placed one next to the other such that 190 by of sequence separated the end of the first PTU's termination sequence and the beginning of the next PTU's promoter sequence. This was achieved by cutting the HBF vector (here, FIG. 6) in two separate reactions. The first reaction cut with PmeI to linearize the vector. The second reaction cut with PmeI and NheI and the NheI 5′ overhang was filled in using a Klenow fragment. This second fragment consisted of spacer DNA sequence, the extended globulin promoter, the HBsAg coding sequence, and the PinII termination sequence. The PmeI/filled NheI fragment was inserted into the linearized vector's PmeI site using a blunt end ligation and the orientation of the fragment was screened using restriction fragment analysis. This resulted in a two PTU vector.

### Example 2

### Transformation and Assays

The HBE construct was transformed into maize plants using an Agrobacterium tumefaciens superbinary vector transformation system. Zea mays was used as the host plant and regeneration of plants were conducted in growth chambers and greenhouses to generate T₀ plants producing T₁ seed. Expression levels for HBE T₁ seed were assayed using a sandwich ELISA protocol as briefly described below:

Extraction of HBsAg from seed: 100 mg of ground seed samples were agitated in 1 mL of extraction buffer (PBS with 1% Triton X-100) with a ball bearing at 600 oscillations per minute for 20 seconds, centrifuged, and the supernatant diluted and applied to the ELISA plate. ELISA assay for HBsAg on 96-well plate: Rabbit anti-HBsAg antibody was used to coat 96-well plates, corn extracts were added to the coated plates, followed by biotinylated rabbit anti-HBsAg, streptavidin conjugated to alkaline phosphatase, and finally pNPP substrate. Washes were conducted between each step using PBST and blocking solution was 3% BSA in PBST.

### Example 3

### Preparation of HBsAg Expressing Lines

All defatting work was done with material derived from HBE lines.

### HBE Lines Used:

CC090013)1: HBE T₀ plants were backcrossed 3 times to SP122 and selfed twice to produce a homozygous line. Seed from the second self were tested for homozygosity using herbicide screening (100% resistance). These plants were grown to maturity and selfed to produce the CC090013)1 seed. The ground seed was then used as a positive control for the ELISA assays.
CC10005411: HBE T₀ plants were backcrossed 6 times to SP114 and selfed once to produce seed for defatting experiments. Whole seed and germ were used for defatting experiments. Germ was extracted from the seed by soaking them in water overnight and hand dissecting the embryo (germ). The germ was then dried overnight at 37° C. to a moisture content of 6-15%.

### Example 4

### Effect of Fat Addition and Fat Reduction on Antibody Detection of HBsAg

Germ and seed of the lines prepared as above were ground in a coffee grinder to the texture of fine cornmeal.

CC100054]1 ground material was defatted using a hexane treatment or a supercritical fluid extraction (SFE) treatment. For hexane treatment, a total of 5 mL of hexane was added to every 1 g of seed or germ material. For SFE treatment, ground germ was defatted using a constant CO₂ flow of 20 g/min, at 350 bar, and 35° C. in the extraction chamber. Conditions were maintained until oil was no longer exiting the waste chamber.

Oil or butter was added to the ground plant material as indicated in the table below.

Extraction of the HBsAg from the ground seed was done as described above, with various extraction buffers (see table below “Extraction Conditions” for details). The concentration of HBsAg in seed and germ was determined using a sandwich ELISA as described above.

It was initially observed that adding 80 uL melted butter to 100 mg ground germ or seed or adding 50 to 80 uL canola oil to 100 mg germ was associated with decreased detection of HBsAg compared to ground germ or seed where oil or butter was not added. Tests were then conducted on defatting ground germ and seed. Results are summarized below.

TABLE 1
Ground seed

| | | Median | |
|---|---|---|---|
| | | HBsAg | |
| Sample | Extraction conditions | (ng/uL) | Data source |
| CC100054]1 | PBS + 0.05% Tween | 0.03 | HepB_CH029 |
| seed | | | |
| CC100054]1 | | 0.11 | |
| hexane | | | |
| defatted seed | | | |
| CC100054]1 | PBS + | 1.42 | HepB_CH029 |
| seed | 0.1% Triton X-100 | | |
| CC100054]1 | | >3 | |
| hexane | | | |
| defatted seed | | | |

TABLE 2
Ground germ

| | | | Median | |
|---|---|---|---|---|
| | | Extraction | HBsAg | |
| Sample | Conditions | conditions | (ng/uL) | Data source |
| CC100054]1 | No oil/butter | PBS + | 4.52 | HepB_CH028 |
| fullfat germ | | 0.1% Triton | | |
| CC100054]1 | | X-100 | 7.43 | |
| hexane | | | | |
| defatted germ | | | | |
| CC100054]1 | No oil/butter | PBS + 0.05% | 0.27 | HepB_CH029 |
| fullfat germ | | Tween | | |
| CC100054]1 | | | 0.34 | |
| hexane | | | | |
| defatted germ | | | | |
| CC100054]1 | No oil/butter | PBS + | 1.89 | HepB_CH029 |
| fullfat germ | | 0.1% Triton | | |
| CC100054]1 | | X-100 | 6.47 | |
| hexane | | | | |
| defatted germ | | | | |

The inventors concluded there was an association between reducing fat content of the plant material and increased detection of the membrane bound protein.

### Example 5

### Antibody Production in Animals in Response to Administration of Plant Produced HBsAg

In an initial experiment (mouse trial #1), whole, full-fat HBE seed was used to produce HBsAg expressing germ and was fed to mice, but an anti-HBsAg immune response could not be detected when compared to whole, full-fat control germ. The seed was soaked and degermed by hand. The germ was maintained at about 50% moisture and stored in the −80 C freezer and thawed just prior to feeding the mice. Mice were initially injected with 1.0 ug Recombivax (commercial HBsAg vaccine) and at nine weeks post-injection fed 5 g of whole germ on three consecutive days. A total of 3 oral boosts were administered, each 2 weeks apart.

The following experimentation used HBE seed and demonstrated that reducing oil in plant material provided for an immune response.

### Mouse Trial #2

Grain containing the HBE construct was collected and degermed by soaking the seed in water 1-3 days and hand dissecting the germ from the endosperm using pliers. Dissected germ was dried in a 37° C. incubator to 6-15% moisture. Germ from transgenic lines as well as a non-transgenic line (control germ) was then ground using a coffee grinder to a fine cornmeal consistency. Both control and HBsAg germ were defatted using a hexane extraction. Every 1 gram of ground germ was extracted with 5 mL of hexane. Residual hexane was removed from the germ by evaporation at room temperature over 1-3 days. All hexane extracted germ batches were thoroughly mixed to create a homogenous antigen concentration.

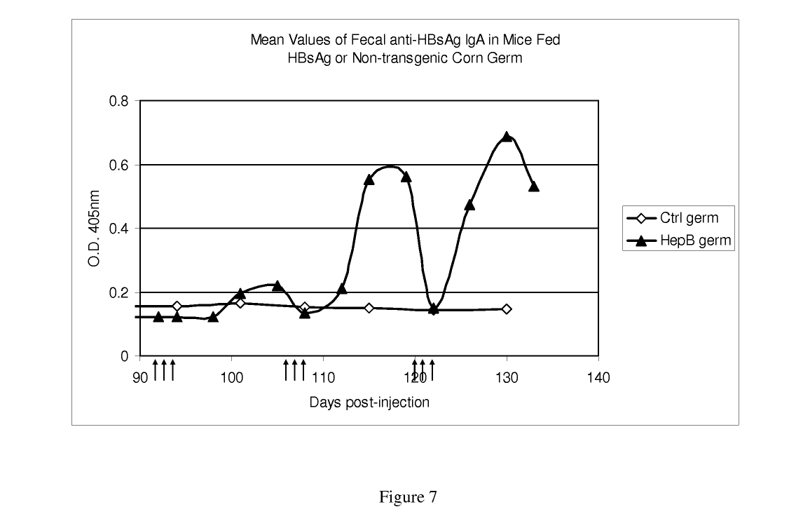

Balb/c mice were injected with 0.5 μg of Recombivax, a commercially available Hepatitis B vaccine, on Day 0. HBsAg or control germ were fed to mice 14 weeks post-injection. Mice received three oral boost doses each two weeks apart. Each oral boost consisted of three 5 g doses administered over three consecutive days (Days 92, 93, 94, 106, 107, 108, 120, 121, 122). The final % TSP in the ground germ was approximately 0.2%, therefore mice were fed approximately 0.6 mg of HBsAg per 5 g oral dose. Fecal samples were collected at the time of initial injection, and then every two weeks following oral boost doses (Days 0, 92, 94, 98, 101, 105, 108, 112, 115, 119, 122, 126, 129, and 133). IgA was detected using an ELISA. Briefly, HBsAg was used to coat ELISA plates. Plates were subsequently incubated with fecal samples (100 mg resuspended in 1 mL of 1×PBS+1% BSA+protease inhibitor), an anti-mouse IgA antibody conjugated to alkaline phosphatase, and a pNPP solution. Reaction O.D.s were read at 405 nm. Results are summarized in FIG. 7. Arrows indicate the oral boost days. With each oral boost, a progressively stronger IgA induction was seen. This established that defatted HBsAg plant material can induce a strong immune response in mice.

### Example 6

Mouse trials are conducted using plant material expressing membrane bound proteins, including HIV gp120 segment of the env gene (GenBank accession U63632) in which codons were changed to reflect optimal codon usage in corn and to eliminate any potential message destabilizing sequences as is described at US Patent Application 20040040061, incorporated herein by reference in its entirety.

What is claimed is:

1. A method of increasing antigenicity of a plant-produced membrane-bound polypeptide, said method comprising,
a) expressing a membrane-bound polypeptide in a plant composition selected from the group consisting of a plant, plant tissue or plant part;
b) reducing fat content of said plant composition;
c) testing said membrane-bound polypeptide expressed in said plant composition having reduced fat content to determine whether said membrane-bound polypeptide has increased antigenicity compared to said membrane-bound polypeptide expressed in said wherein said plant composition does not having reduced fat content; and
d) providing said membrane-bound polypeptide with increased antigenicity expressed in a plant composition having reduced fat content.

2. The method of claim 1, wherein said testing comprises contacting said membrane-bound polypeptide expressed in said plant composition having reduced fat content with an antibody specific for said membrane-bound polypeptide and determining if said membrane-bound polypeptide expressed in said plant composition having reduced fat content binds to a higher degree to said antibody, compared to binding of said membrane-bound polypeptide to said antibody when expressed in said plant composition not having reduced fat content.

3. The method of claim 1, wherein said testing comprises administering said membrane-bound polypeptide expressed in said plant composition having reduced fat content to an animal and determining if there is an increased protective immune response in said animal compared to administration of said membrane-bound polypeptide when expressed by said plant composition not having reduced fat content.

4. The method of claim 1, wherein said fat content of said plant composition is reduced after introducing said membrane-bound polypeptide into said plant.

5. The method of claim 1, further comprising extracting said membrane-bound polypeptide from said plant composition.

6. The method of claim 1, wherein said plant composition comprises seed.

7. The method of claim 1, wherein said plant composition comprises germ.

8. The method of claim 7, further comprising separating said germ from seed of said plant.

9. The method of claim 1, wherein said membrane-bound polypeptide comprises hepatitis B surface antigen.

10. A method of producing an increased antigenic response in an animal to a plant-produced membrane-bound polypeptide, said method comprising,
a) expressing a membrane-bound polypeptide in a plant composition selected from the group consisting of a plant, plant tissue or plant part;
b) reducing fat content of said plant composition; and
c) administering said membrane-bound polypeptide to said animal such that said membrane-bound polypeptide expressed in said plant having reduced fat content produces an increased antigenic response in said animal compared to administering said membrane-bound polypeptide when expressed in said plant wherein said plant does not have reduced fat content.

11. The method of claim 10, wherein said membrane-bound polypeptide protects an animal from a disease and wherein said animal has an increased protective response to said disease when administered said membrane-bound polypeptide expressed in said plant composition having reduced content, compared to an animal administered said membrane-bound polypeptide expressed in said plant not having reduced fat content.

12. The method of claim 10, wherein said animal administered said membrane-bound polypeptide expressed in said plant having reduced fat content produces antibodies binding to a higher affinity to said polypeptide expressed in said plant having reduced fat content, compared to an animal administered said polypeptide expressed in said plant not having reduced fat content.

13. The method of claim 10, wherein said plant composition comprises seed.

14. The method of claim 10, wherein said plant composition comprises germ.

15. The method of claim 14, further comprising separating said germ from seed of said plant.

16. The method of claim 10, wherein said membrane-bound polypeptide is extracted from said plant composition.

17. The method of claim 10, wherein said plant composition is administered to said animal.

18. The method of claim 10, wherein said membrane-bound polypeptide comprises hepatitis B surface antigen.


SEQUENCE LISTING (12 sequences)

SEQ ID NO: 1 - DNA, 750 bp, Artificial Sequence
  Description of Artificial Sequence Synthetic polynucleotide
atggccaacaagcacctgagcctctccctcttcctcgtgctcctcggcctctccgcctccctcgccagcggcgagtccaccacctccggcttcctcggcccgctcctcgtgctccaggccggcttctccctcctcacccgcatcctcaccatcccgcagtccctcgactcctggtggacctccctcaacttcctcggcggcgccccgacctgcccgggccagaacctccagtccccgacctccaaccactccccgacctcctgcccgcccacctgcccgggctaccgctggatgtgcctccgccgcttcatcatcttcctcttcatcctcctgctctgcctcatcttcctcctcgtgctcgtggactaccagggcatgctcccggtgtgcccgctcctcccgggcacctccacgacctccaccggcccgtgcaagacctgcaccatcccggcccagggcacctccatgttcccgtcctgctgctgcaccaagccgtccgacggcaactgcgcctgcatcccgatcccgtcctcctgggccttcgcccgcttcctctgggagtgggcctccgtgcgcttctcctggctctccctcctcgtgccgttcgtgcagtggttcgtgggcctctccccgaccgtgtggctctccgtgatctggatgatgtggtactggggcccgtccctctacaacatcctctccccgttcctcccgctcctcccgatcttcttctgcctctgggtgtacatctga

SEQ ID NO: 2 - DNA, 675 bp, Hepatitis B virus
gagtccaccacctccggcttcctcggcccgctcctcgtgctccaggccggcttctccctcctcacccgcatcctcaccatcccgcagtccctcgactcctggtggacctccctcaacttcctcggcggcgccccgacctgcccgggccagaacctccagtccccgacctccaaccactccccgacctcctgcccgcccacctgcccgggctaccgctggatgtgcctccgccgcttcatcatcttcctcttcatcctcctgctctgcctcatcttcctcctcgtgctcgtggactaccagggcatgctcccggtgtgcccgctcctcccgggcacctccacgacctccaccggcccgtgcaagacctgcaccatcccggcccagggcacctccatgttcccgtcctgctgctgcaccaagccgtccgacggcaactgcgcctgcatcccgatcccgtcctcctgggccttcgcccgcttcctctgggagtgggcctccgtgcgcttctcctggctctccctcctcgtgccgttcgtgcagtggttcgtgggcctctccccgaccgtgtggctctccgtgatctggatgatgtggtactggggcccgtccctctacaacatcctctccccgttcctcccgctcctcccgatcttcttctgcctctgggtgtacatc

SEQ ID NO: 3 - DNA, 72 bp, Hordeum vulgare
atggccaacaagcacctgagcctctccctcttcctcgtgctcctcggcctctccgcctccctcgccagcggc

SEQ ID NO: 4 - DNA, 1401 bp, Zea mays
aagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaa

SEQ ID NO: 5 - DNA, 1386 bp, Zea mays
aagcttgccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttctaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgc

SEQ ID NO: 6 - DNA, 15 bp, Zea mays
atacagccaacccaa

SEQ ID NO: 7 - DNA, 3006 bp, Zea mays
cggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactacacgatg

SEQ ID NO: 8 - DNA, 2950 bp, Zea mays
cggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgc

SEQ ID NO: 9 - DNA, 53 bp, Zea mays
atacagccaacccaacacacacccgagcatatcacagtgacagacactacacg

SEQ ID NO: 10 - DNA, 3006 bp, Zea mays
cggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactacaccatg

SEQ ID NO: 11 - DNA, 43 bp, Zea mays
ggatccaacacacacccgaggatatcacagtcgacactacacc

SEQ ID NO: 12 - protein, 4 aa, Unknown
  Description of Unknown “KDEL” motif peptide
KDEL